(19) United States
(12) Patent Application Publication
(10) Pub. No.: US 2015/0360222 A1
(43) Pub. Date: Dec. 17, 2015

(54) Sampling Device for Fluids

(71) Applicant: Stefan DANNELÖV, (US)

(72) Inventor: Stefan DANNELÖV, Umeå (SE)

(21) Appl. No.: 14/443,111

(22) PCT Filed: Feb. 5, 2014
(86) PCT No.: PCT/SE2014/000014

§ 371 Date: May 15, 2015

(30) Foreign Application Priority Data
Mar. 5, 2013 (SE) 1300167-2

(51) Int. Cl. B01L 3/00; G01N 1/10; A61L 2/28

(52) U.S. Cl. CPC B01L 3/508; A61L 2/28; G01N 1/10; B01L 2200/028; B01L 2300/0832

(57) ABSTRACT

Device for sampling (1) liquid comprised of at least one collection part (2), with at least one liquid collection surface (4) and at least one sampling container (3), with at least one inner chamber (6) which are intended to be temporarily connected to each other in conjunction with the sampling of liquid by the collection part (2) being partially inserted into the sampling container (3). The collection part (2) includes at least one tubular shaped conveying member (5) with a first outfall (8), which opens onto the collecting surface (4) and a second outfall (9), which when the collection part (2) and the sampling container (3) are connected to each other, open into the inner chamber (6) at a level lower than half the height of the height between the inner chamber's (6) top edge (10) and bottom (11).

### FIELD OF THE INVENTION

The present invention concerns a sampling device for fluids (liquids) in accordance with the claims.

### BACKGROUND OF THE INVENTION

There exist several problems in conjunction with the cleansing and/or disinfection of medical instruments. One of the problems associated with the cleaning and disinfection of medical instruments consist of ensuring that a satisfactory result of the cleaning or disinfection is achieved. To ensure this, some kind of examination (test) may be performed, each time or regularly, on the surface of the instruments that have been processed, so that proper cleanliness and disinfection is guaranteed for all instruments treated or that it may be determined that the instruments are not unclean. An example of one type of examination consists of swabbing a single instrument on its surface with one of the following methods either protein or ATP. This is however difficult to perform on hollow instruments with small lumen.

Another way to determine an instruments degree of hygiene is to test the final rinse water. The degree of purity of the final rinse water has in empirical tests proved to be coupled to the final results of the cleaning process. One way to determine if the proper cleaning procedure was performed is to investigate whether there are traces of impurities, proteins, cells, bacteria, or similar in the rinse water, specifically the final (last) rinse water. This may be accomplished by sampling the rinse water. Hollow instruments will also be included in the examination when tests are performed on the rinse water.

One problem associated with the sampling of rinse water is to ensure that the sample taken is relevant, i.e. representative of the rinsing liquid, that is to say that the rinse water consists essentially of the last rinsing water used in the procedure. There is a risk that traces of previous rinse water and also that which is to be cleaned away is left in the sampling container. To eliminate this occurrence, equipment has been developed that prevents rinse water from becoming stationary in the sampling container, this is accomplished by capturing larger amounts of rinse water and letting it run down to the bottom of the sampling container and thereby greatly increase the circulation in the sampling container.

Moreover, there is a problem in the performance of third-party testing of equipment that includes a sampling function. In these cases, the equipment's automatic function for sampling the rinse water is not used. For third-party testing, the equipment's normal sampling function can not be used because these devices are integrated into the equipment and thereby can not be used for third-party analysis because it would not be unbiased.

A further problem is to create a reliable design which is substantially more inexpensive to produce. In a number of contexts, there is a need to take samples or similar of a liquid or the like used in some type of process or the like.

The purpose of the present invention is to solve at least one of the above mentioned problems. The purpose is accomplished with a design in accordance with the claims.

### PRIOR ART

US20100202920 describes a variant of a device and method for sampling of washing, disinfecting and rinsing liquids in a machine intended to be used for disinfecting instruments and the like. The device and the method differ greatly from the design of the present patent application. For example, the device in the machine consists of one in the machine integrated function. It is not possible to perform a third-party investigation of the machine with the device.

U.S. Pat. No. 6,536,060 describes a variant of a method and a device which are intended to be used for monitoring a cleaning process inside a washing machine. The design includes a cup-like collection device in which fluid is collected and analyzed. The method and device in accordance with its description differ to a significant extent from the design in accordance with the present patent application.

U.S. Pat. No. 7,150,284 describes a variant of a device for monitoring a cleaning process. The device includes a function for sampling the used liquid. The device differs significantly from the design of the present patent application.

U.S. Pat. No. 7,470,404 describes a variant of a cup which is intended to be used for collecting and sampling a fluid. The design includes a cylindrical inner container which is inserted down into an outer container from which the sample is taken. During sampling a valve is opened between the cylindrical container and the outer container. The design differs to a significant extent from the design in accordance with the present invention.

### BRIEF DESCRIPTION OF THE DRAWINGS

In the following detailed description of the present invention, reference and references to the following figures will occur. These figures are briefly described in the following figure list.

FIG. 1A shows a first embodiment of the present invention in perspective askew from above.

FIG. 1B shows a first embodiment of the present invention from a side-view with hidden contour lines.

FIG. 1C shows a first embodiment of the present invention from a top-view with hidden contour lines.

FIG. 2A shows the sampling container in perspective askew from above.

FIG. 2B shows the sampling container from a side-view.

FIG. 2C shows the sampling container in a view from above.

FIG. 3A shows the collecting part in a view from above.

FIG. 3B shows the collecting part in a view from the side.

FIG. 3C shows the collecting part from a second side-view.

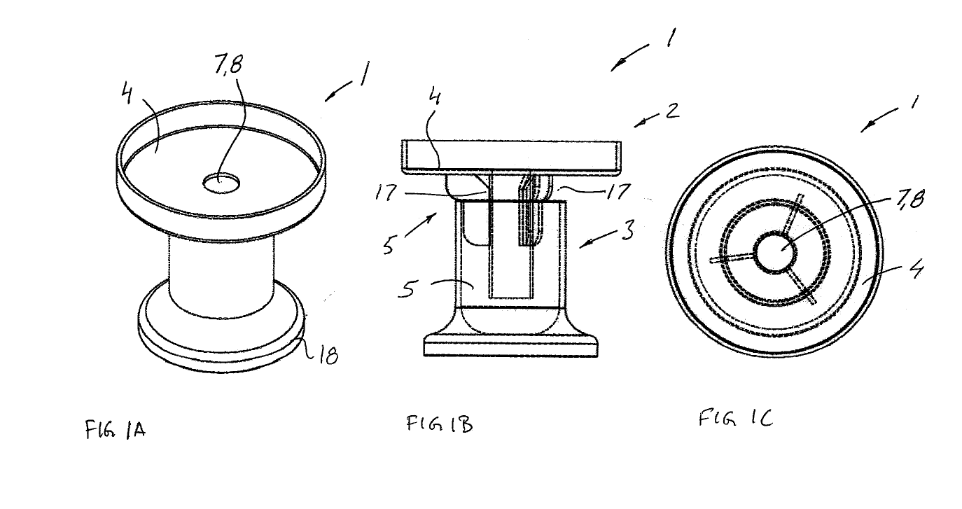

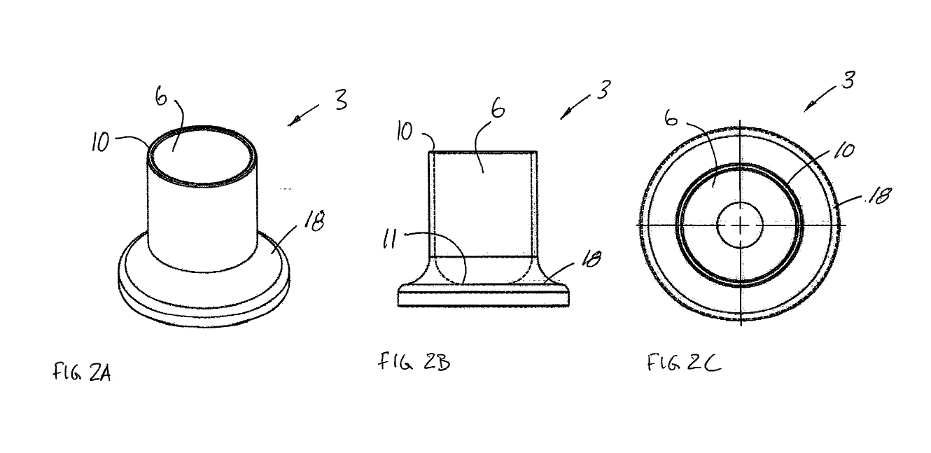

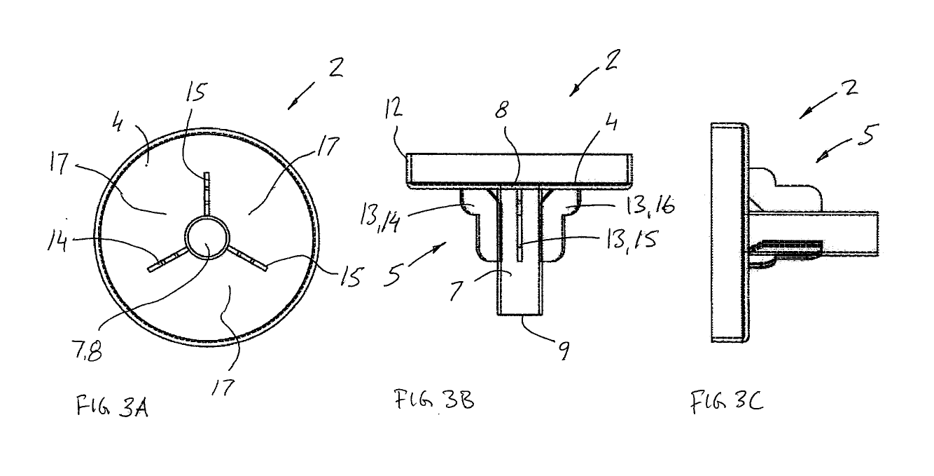

### DETAILED DESCRIPTION OF THE INVENTION

With reference to the figures, a sampling device 1 in accordance with the present invention is shown. The present sampling device 1 is preferably intended to be used in the sampling of liquid, such as preferably rinsing liquid in a dishwasher for instruments and the like. The collected liquid is preferably the final rinsing liquid. Despite the fact that a specific application of the present invention is indicated, it may be used in other applications where it is suitable to use. Thus, the present invention is not limited to use in connection with the sampling rinsing liquids.

A sampling device 1 for liquids comprised of at least one collection part 2 and at least one sampling container 3. The collection part 2 and the sampling container 3 are, in conjunction with sampling, intended to be temporarily connected to each other by the collection part 2 being partially inserted into the sampling container 3.

The collection part 2 is comprised of at least one liquid collecting surface 4 and at least one conveying member 5 by which fluid is conveyed to at least one inner chamber 6 in the sample container 3. The conveying member 5 is preferably tubular and includes at least one channel 7 whose first outfall 8 opens onto the collecting surface 4 and a second outfall 9, which is when the collection part 2 and sampling container 3 are connected to each other opens into the inner chamber 6 in the sampling container 3 at a level less than half the height between the inner chamber's 6 top edge 10 and bottom 11.

When the collection part 2 and the sampling container 3 are connected to each other, the conveying member's 5 outfall 9 opens into the inner chamber 6 of the sampling container 3 at a level lower than a quarter of the height between the inner chamber's 6 top edge 10 and the bottom 11.

When the collection part 2 and the sampling container 3 are connected to each other, the conveying member's 5 outfall 9 opens into the inner chamber 6 of the sampling container 3 at a level lower than a fifth of the height between the inner chamber's 6 top edge 10 and the bottom 11.

In the exemplifying embodiment of the present invention, the liquid collecting surface 4 is essentially flat (plane) and surrounded by at least one rim 12, edge (boundary) or the like which allows for the collected liquid to achieve a specific depth. The liquid collecting surface's 4 shape in alternative embodiments may deviate from the essentially radial direction (an essentially horizontal surface when used). The liquid collecting surface 4 may for instance be inclined in the direction towards the first outfall (and the center of the surface). The inclined liquid collecting surface's 4 shape provides the surface with a liquid draining effect. It is further conceivable that the rim's height is adjustable, accomplished for example by connectable rings or other suitable for the purpose devices. The transition between the surface and the rim preferably has a shape which facilitates cleaning and prevents residue (deposits) from remaining in the corner. It is further conceivable that the liquid collecting device includes several different collection parts with different liquid collection and liquid conveying properties.

In different embodiments of the present device 1, the cross-sectional area of the conveying member's channel is less than 80 percent of the cross-sectional area of the internal space of the sampling container. In the preferred embodiment of the present device, the cross-sectional area of the conveying member's channel is between 10 to 30 percent of the cross-sectional area of the inner chamber 6 of the sampling container 3. The stated area ratios between the channel's cross-sectional area and the inner chamber's cross-sectional area do not exclude other area ratios.

In the exemplifying embodiment, the collection part 2 and sampling container 3 have round cross-sections. In alternative embodiments it is conceivable that at least one of either the collection part 2 or the sampling container 3 has another for the purpose suitable form (shape) or forms. It is thus conceivable that the collection part 2 and/or the sampling container 3 have an oval or other non-round cross-section. It is further conceivable that the collection part 2 and the sampling container 3 have other cross-sectional shapes such as triangular, square, pentagonal and other multiple edges.

The collection part 2 preferably includes a spacer unit 13 which allows for the creation of a certain distance between the sampling container 3 and the collection part 2. The spacer unit 13 further has the technical effect of constituting a centering device for the collection part's 2 position relative to the sampling container 3. In the exemplifying embodiment, the spacer unit 13 is comprised of at least one first distance and centering member 14, at least one second distance and centering member 15 and at least one third distance and centering member 16. In alternative embodiments, the number of distance and centering members may be more or less than three. In alternative embodiments it is conceivable that the spacer unit's length is adjustable. This adjustability may be achieved by the design including at least one first tube and at least one second tube which are arranged to be movable or with another for the purpose of suitable technology.

Between the centering member in the collection part 2 and the rim 12 of the sampling container 3, at least one channel 17 bushing, intermediate space or the like is formed through which the flow of fluid from the inner chamber 6 may occur. Preferably, multiple channels 17 or the like are formed.

The sampling container 3 preferably includes at least one inner chamber 6, to which the liquid collected in the collection part is intended to be conveyed to via conveying member 5. The inner chamber 6 preferably has such a volume that culturing may take place. In the exemplifying embodiment the container holds about 50 ml. In alternative embodiments, the inner chamber may have capacity for another for the purpose suitable volume greater or less than approximately 50 ml.

The sampling container 3 in the exemplifying embodiment also includes a variant of a foot 18, base support unit or the like. The design of the foot may vary greatly within the scope of the present invention.

In the preferred embodiment, the transition between the inner chamber's wall and bottom is radius-shaped or otherwise curved. It is further conceived that the bottom has an angle other than 90 degrees relative to the axial direction of the collection part. The interior surface or interior surfaces should be of such a form which permits easy cleaning of the design. The interior surfaces' lower edge must also be of a form that allows a good flow that enables efficient exchange of liquid in the reservoir. This reduces the risk of error in measurement and that the sample is a representative sample of the final rinsing liquid. The liquid collecting surface may be straight, curved, or consist of at least one combination of said forms.

In alternative embodiments it is conceivable that the sampling container 3 consists of one or more parts. These parts are in such cases joined with an appropriate attachment method. It is thus conceivable that the sampling container 3 consists of at least two parts which are joined together in a manner suitable for the purpose such as for example by welding or a similar method (process).

The material of the sampling device's 1 parts consist preferably of stainless steel or the like. In alternative embodiments, however, it is conceivable that the sampling device is made of another for the purpose suitable materials or combinations of materials. It is further conceivable that the sampling device's parts are made of different types of materials.

In alternative embodiments, it is conceivable that the sampling device at the transition between the collection part and the sampling device include at least one protection safeguard, protective shield, to prevent the spray of water from the side penetrating into the container through for example the underside of the sampling device.

When using the present sampling device, it is placed in the machine (equipment) in which a sample is to be taken. The sampling device may be advantageously placed together with other goods and be part of the process chain as part of the goods to be processed.

In alternative embodiments, it is conceivable that the present sampling device for liquids includes interchangeable collection parts. For example, the device may be included in a system for sampling which includes at least one first sampling container, a first collection part and at least one second collection part. The collection parts may have different fluid collection and fluid conveying properties. With this system the liquid collection and liquid conveying abilities of collecting part (of the sampling device) may be adapted to different machines (equipment).

In the detailed description of the present invention, design details may have been omitted which are apparent to persons skilled in the art in the field of the device. Such obvious design details are included to the extent necessary so that the proper and full performance of the present invention is achieved.

Even if certain preferred embodiments have been described in detail, variations and modifications of the method and the device may become apparent for specialists in the field of the invention. All such modifications and variants are regarded as falling within the scope of the following claims.

### Advantages of the Invention

Several advantages are achieved with the present invention. Firstly, an improved device for sampling is achieved which solves at least one of above mentioned technical problems. For example, the device allows for an enhanced ability to take a representative sample of the final rinse water in a washing (cleaning) machine (apparatus). Another advantage of the present invention is that third-party sampling of an existing machine (equipment) may occur.

1. A device for sampling (1) liquid comprised of at least one collection part (2), with at least one liquid collection surface (4) and at least one sampling container (3), with at least one inner chamber (6) which are intended to be temporarily connected to each other in conjunction with the sampling of liquid by the collection part (2) being partially inserted into the sampling container (3) characterized by that the device includes at least one spacer unit (13) which forms a distance between the sampling container's (3) top edge (10) and the collection part's (2) liquid collection surface (4) and that the collection part (2) includes at least one tubular shaped conveying member (5) with a first outfall (8), which opens onto the collection surface (4), and a second outfall (9), which when the collection part (2) and the sampling container (3) are connected to each other, open into the inner chamber (6) at a level lower than half the height of the height between the inner chamber's (6) top edge (10) and bottom (11).

2. A device for sampling (1) in accordance with claim 1 and characterized by that the second outfall (9), when the collection part (2) and the sampling container (3) are connected to each other, opens into the inner chamber (6) at a level lower than a quarter of the height between the inner chamber's (6) top edge (10) and bottom (11).

3. A device for sampling (1) in accordance with claim 1 and characterized by that the second outfall (9), when the collection part (2) and the sampling container (3) are connected to each other, opens into the inner chamber (6) at a level lower than a fifth of the height of the height between the inner chamber's (6) top edge (10) and bottom (11).

4. A device for sampling (1) in accordance with claim 1 and characterized by that the distance between the collection part's (2) liquid collection surface and the sampling container's (3) top edge (10) are arranged to be adjustable via at least one adjustment device.

5. A device for sampling (1) in accordance with claim 1 and characterized by that the spacer unit (13) includes at least one first distance and centering member (14), at least one second distance and centering member (15) and at least one third distance and centering member (16).

6. A device for sampling (1) in accordance with claim 1 and characterized by that the liquid collecting surface (4) is surrounded by at least one rim (12).

7. A device for sampling (1) in accordance with claim 1 and characterized by that the liquid collecting surface (4) is inclined in the direction toward the first outfall (8).

8. A device for sampling (1) in accordance with claim 1 and characterized by that the cross-sectional area of the conveying member is less than 80 percent of the cross-sectional area of the internal space of the sampling container.

9. A device for sampling (1) in accordance with claim 1 and characterized by that the cross-sectional area of the conveying member equals between 10 to 30 percent of the cross-sectional area of the internal space of the sampling container.

10. A device for sampling (1) in accordance with claim 1 and characterized by that the collection part (2) is interchangeable.
